(19) United States
(12) Patent Application Publication
(10) Pub. No.: US 2023/0115466 A1
(43) Pub. Date: Apr. 13, 2023

(54) APPARATUS AND METHODS FOR BARIATRIC SURGERY

(71) Applicants: Simcha Milo, Haifa (IL); Ehud Milo, San Mateo, CA (US)

(72) Inventors: Simcha Milo, Haifa (IL); Ehud Milo, San Mateo, CA (US)

(21) Appl. No.: 17/947,061

(22) Filed: Sep. 16, 2022

Related U.S. Application Data
(60) Provisional application No. 63/245,170, filed on Sep. 16, 2021.

(51) Int. Cl. A61F 5/00; A61B 17/064

(52) U.S. Cl. CPC A61F 5/0083; A61B 17/064; A61B 2017/00477

(57) ABSTRACT

A pre-assembled chain of staples is provided that is suitable for use in bariatric surgery. The pre-assembled chain of staples includes a plurality of staples, each of the staples having two spaced apart legs and a crown connector which joins the two legs to each other at upper ends thereof and spaces the two legs a predetermined fixed distance apart from each other at their upper ends. The chain of staples also includes rings for joining adjacent staples together, which rings can be separate from the staples.

### CROSS REFERENCE TO RELATED APPLICATION

This application claims benefit from Provisional Application No. 63/245,170, filed Sep. 16, 2021, which is hereby incorporated herein by reference in its entirety.

### FIELD

Apparatus and methods for bariatric surgery are described herein, and, in particular, interlocking staples and methods for their use in bariatric surgery.

### BACKGROUND

Bariatric surgery is often performed to help obese patients lose weight. A typical process can include inserting staples in a pair of parallel groups, with each group having multiple lines of staples, to enable devising between the pair of parallel groups.

More specifically, simple staples lined up in three rows are ejected into the gastric tissues in two parallel groups to enable devising between them. Several such repeated steps, enable separating and or removing sections from the stomach, or of the gastrointestinal tract or similarly of lung sections and alike. Single metal staples are lined up inside a magazine inserted into one of the jaws of the stapling device and by applying pressure, deployed into the tissues while the distal parts of the staples meet the counter jaw of the stapler which acts as an anvil, and are bent by the anvil to secure anchoring. Similar apparatus and methods are used not only for straight lines deployments but also for circular anastomoses. In both applications, leakage and resulting complications can be problematic.

### BRIEF DESCRIPTION OF THE DRAWINGS

FIG. 1 is a perspective view of a pre-assembled chain of staples, where a ring is attached to one of the legs of each of the staples and the leg of an adjacent staple is inserted into the ring.

FIG. 2 is a perspective view of the chain of staples of FIG. 1 inserted into tissue in a line and next to adjacent lines of staples.

FIG. 3 is a perspective view of a pre-assembled chain of staples, where a circular ring is used to secure adjacent legs of adjacent staples together.

FIG. 4 is a perspective view of the chain of staples of FIG. 3 inserted into tissue in a line and next to adjacent lines of staples.

FIG. 5 is a perspective view of a pre-assembled chain of staples, where a race-track shaped ring is used to secure adjacent legs of adjacent staples together.

FIG. 6 is a perspective view of the chain of staples of FIG. 5 inserted into tissue in a line and next to adjacent lines of staples; and

FIG. 7 is a perspective view of an alternative chain of staples.

### DESCRIPTION

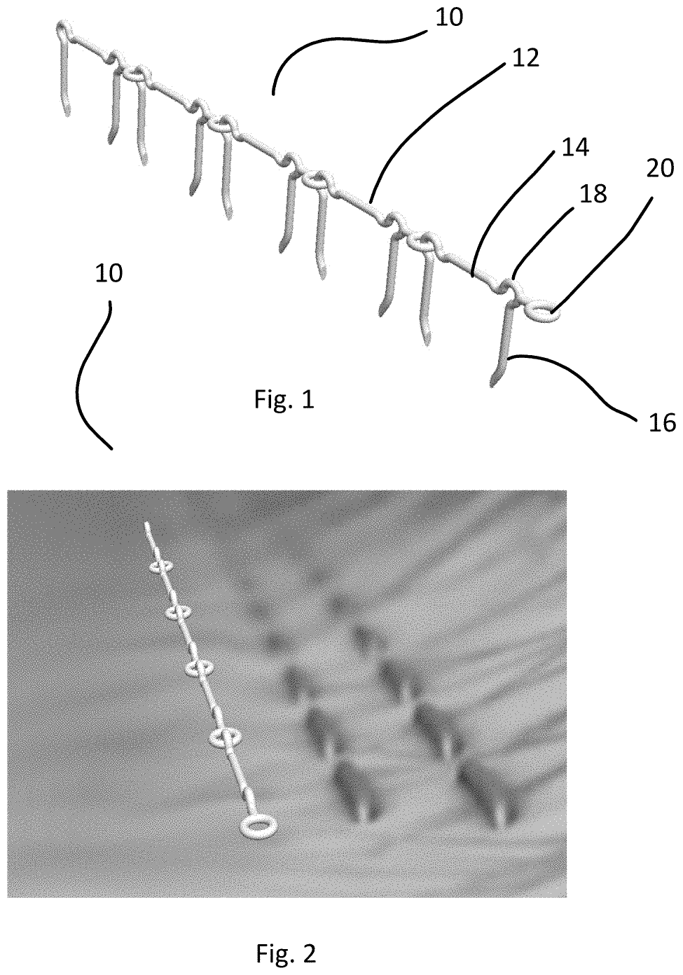

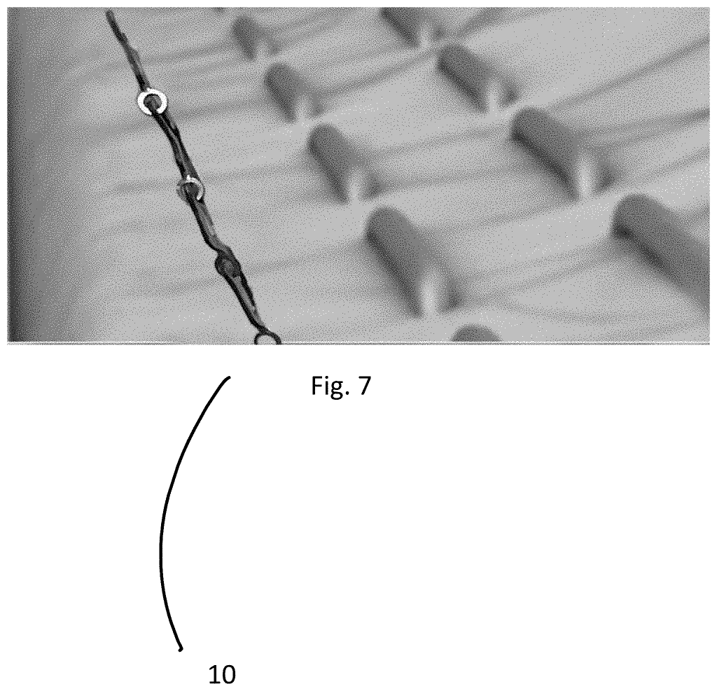

A pre-assembled chain of staples is described herein that is suitable for use in bariatric surgery. Exemplary embodiments of the chain of staples are shown in FIGS. 1-7. By providing the staples in a pre-assembled chain, the spacing between adjacent staples can be more accurately controlled to thereby fixating the distance between the single staples that will withstand intraluminal pressure, residual gastric, intestinal and alike, from enabling the creation of passages between individual staples and staple rows, and reduce the likelihood of leakage.

To improve surgical results and, more specifically, to reduce or eliminate postoperative leakage from and nearby the resulting gastric, intestinal, or other organs stapling line, which is done nowadays by several lines of individual staple, the use of interlocking staples in the form of a chain of staples can be used. Since a large part of the leakage happens secondary to widening of the distances between the individual staples and staple lines under the intra gastric pressure, this cause is expected to be prevented by the act of the staple interlocking, such as being formed in a pre-assembled chain prior to the surgery. Optionally, the stapler magazine can be loaded with an already interlocking chain of staples with the staple legs straight or otherwise spread apart and ready for insertion into tissue.

As shown in FIGS. 1-7, the pre-assembled chain of staples 10 includes a plurality of staples 12, each of the staples 12 having two spaced apart legs 16 and a crown connector 14 which joins the two legs 16 to each other at upper ends thereof and spaces the two legs 16 a predetermined fixed distance apart from each other at their upper ends. Optionally, the legs 16 have a substantially constant cross section in the region above their lower ends which are pointed. The staple 12 is designed to allow said two legs 16 to become bent toward each other once the pointed lower ends have penetrated the tissue surface so as to assume a curved configuration and gather tissue below said surface and constrict same as a part of the pre-assembled chain of staples 10. This can be accomplished by using the very same metals as are in use today or also by a shape memory material for the staples, such as nitinol as are intended for cardiac surgery. The chain of staples 10 also includes a plurality of connection elements, such as rings 20, 22 or 24, for joining adjacent staples 12 together.

The connector 14 includes a central bar, preferably but not necessarily linear, that is preferably, though not necessarily, flanked by a pair of ears 18 configured for receiving the rings 20, 22 or 24. The ears 18 can help retain the rings 20, 22 or 24 and restrict the rings 20, 22 or 24 from sliding down the legs 16. The ears 18 are loop sections of about, e.g., 270 degrees each that may be considered to constitute parts of both the connector 14 and the legs 16. The ring's width should be allowed to fit into the ears 18. The passage of the ring 20, 22, or 24 into the ear 18 should be snug however, requiring little grade of force to keep the unit in the chain 10, together. This can facilitate an easy loading of the needed chain section 10 onto the magazine of a stapler.

In one form, shown in FIGS. 1 and 2, the rings 20 can be attached to one of the staples 12 and positioned so that the leg 16 of an adjacent staple 12 can be inserted into the ring 20. An example of such a staple is disclosed in U.S. Pat. No. 8,475,491, which is hereby incorporated by reference in its entirety.

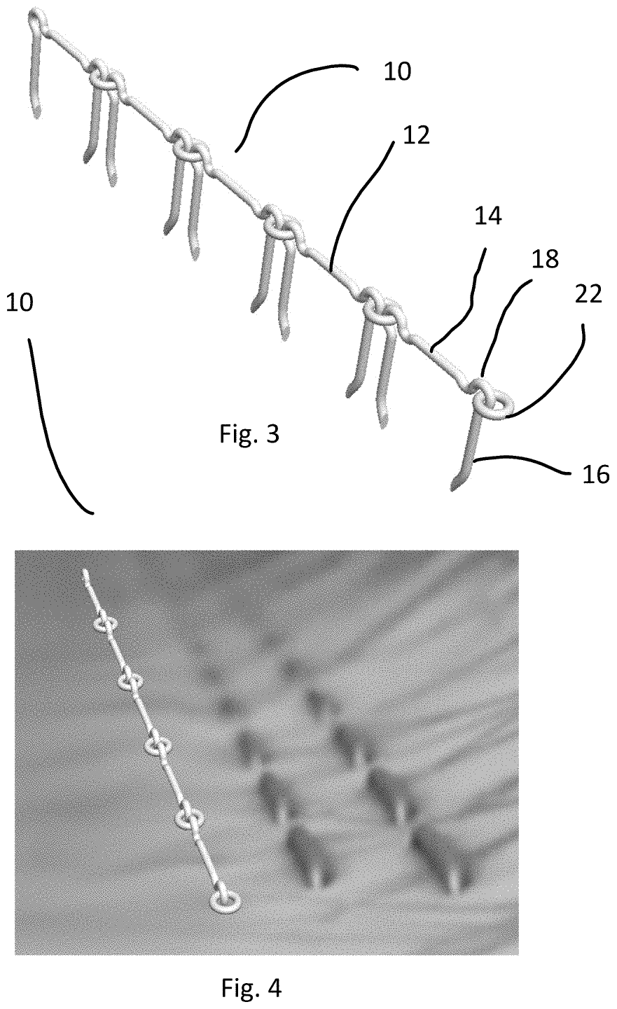

In another form, shown in FIGS. 3 and 4, the rings 22 can be separate from the staples 12, and sized for receiving adjacent legs 16 of an adjacent pair of the staples 12 to restrict movement of the adjacent legs 16 of the adjacent pair of the staples 12 away from each other so that the chain of staples 10 is held together by the rings 22.

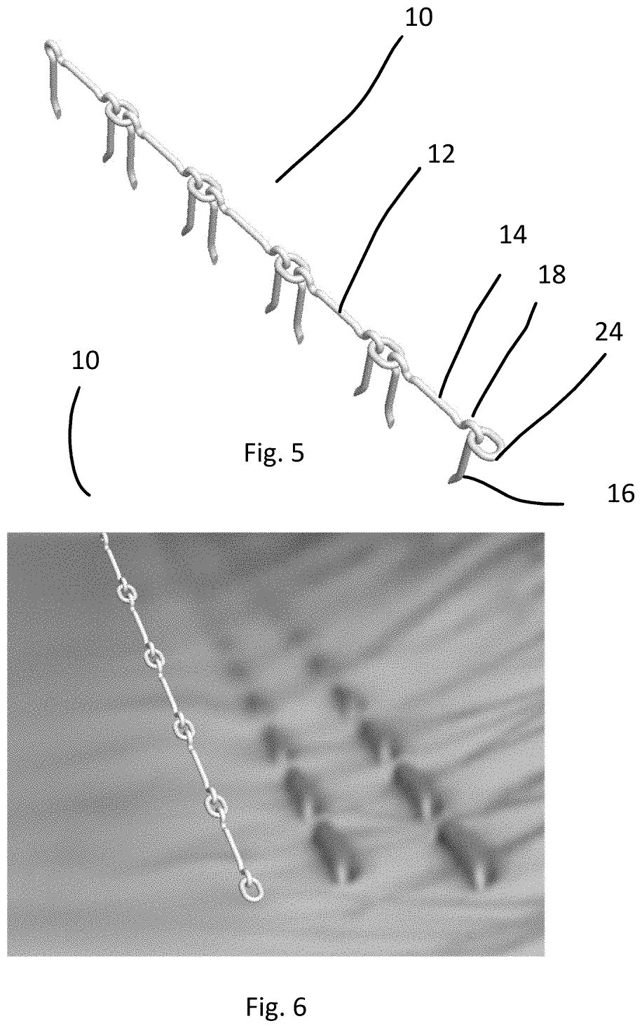

The rings 22 can have a size such that there is a relatively right fit between adjacent staple legs 16, e.g., minimal gaps between the adjacent legs 16 when in the ring 22, as shown in FIGS. 3 and 4. The rings 22 can be circular or round, for example. In the case of a circular ring 22, the inner diameter of the ring 22 should be a little over the sum of the diameters of the legs 16 of two adjacent staples 12. Alternatively, the rings 24 can have a size such that there is a looser fit between adjacent staple legs 16, e.g., a more substantial gap being possible—but not required once inserted into tissue—between the adjacent legs 16 when in the ring 24, as shown in FIGS. 5 and 6. The rings 24 can be race-track or oval shaped, for example. Different types of rings can be used on the same chain of staples 10, e.g., tight-fighting rings 22 with some looser-fitting rings 24.

A method is provided for inserting a row of staples into tissue as part of a surgery, such as a bariatric surgery. The method can include providing the pre-assembled chain 10 described herein. The method can further comprise inserting, preferably but not necessarily sequentially, the pointed ends of each of the legs 16 of the staples 12 of the pre-assembled chain of staples 10 into the tissue.

The method may include allowing the legs 16 of each of the staples 12 to become bent toward each other so as to assume a curved configuration and gather tissue below said surface and constrict the same as a part of the pre-assembled chain of staples 10.

The method may include restricting lengthening of the chain of staples 10, such as by using the rings 20, 22 or 24. The row of staples 12 can be in a line or a curve or circular.

1. A pre-assembled chain of staples suitable for use in bariatric surgery, the chain of staples comprising:
a plurality of staples, each of the staples having two spaced apart legs and a crown connector which joins the two legs to each other at upper ends thereof and spaces the two legs a predetermined fixed distance apart from each other at their upper ends, said legs having a substantially constant cross section in the region above their lower ends which are pointed, said staple being designed to allow said two legs to become bent toward each other once the pointed lower ends have penetrated the tissue surface so as to assume a curved configuration and gather tissue below said surface and constrict same as a part of the pre-assembled chain of staples; and
a plurality of rings, each of the rings configured for receiving adjacent legs of an adjacent pair of the staples to restrict movement of the adjacent legs of the adjacent pair of the staples away from each other so that the chain of staples is held together by the rings.

2. The pre-assembled chain of staples of claim 1, wherein at least some of the rings are sized so that movement of the adjacent legs of the adjacent pair of staples is minimized.

3. The pre-assembled chain of staples of claim 1, wherein at least some of the rings have a racetrack or ovular shape.

4. The pre-assembled chain of staples of claim 1, wherein at least some of the rings have a round shape.

5. A method of inserting a row of staples into tissue as part of a surgery, such as a bariatric surgery, the method comprising:
providing the pre-assembled chain of staples of claim 1; and
inserting, preferably sequentially, the pointed ends of each of the legs of the staples of the pre-assembled chain of staples into the tissue.

6. The method of claim 5, further comprising allowing the legs of each of the staples to become bent toward each other so as to assume a curved configuration and gather tissue below said surface and constrict same as a part of the pre-assembled chain of staples.

7. The method of claim 6, further comprising restricting lengthening of the chain of staples.

8. The method of claim 7, wherein the step of restricting lengthening of the chain of staples further comprises using the rings to restrict lengthening of the chain.

9. The method of claim 5, wherein the row of staples is a line.

10. The method of claim 9, wherein the row of staples is curved or circular.

11. The pre-assembled chain of staples of claim 1, wherein the connector includes a central bar, preferably linear, that is flanked by a pair of ears configured for receiving the rings.
